(19) United States
(12) Patent Application Publication
(10) Pub. No.: US 2026/0146841 A1
(43) Pub. Date: May 28, 2026

(54) Apparatus, Methods and Systems for Fail Safe in Optical Coherence Tomography

(71) Applicant: Canon U.S.A., Inc., Melville, NY (US)

(72) Inventors: Raymond Dawson, Hampton Roads, VA (US); James Bupp, Boston, MA (US); Michael Wilt, Windham, NH (US)

(21) Appl. No.: 18/961,105

(22) Filed: Nov. 26, 2024

(51) Int. Cl. G01B 9/02091; A61B 5/00; G01B 9/02

(52) U.S. Cl. CPC G01B 9/02091; G01B 9/02049; A61B 5/0066; A61B 5/0084

(57) ABSTRACT

Apparatus, methods and systems relating to fluorescence imaging, and more particularly, to reducing or eliminating injuries by a catheter in fluorescence spectroscopy systems, as well as optical coherence tomography (OCT) systems.

### FIELD OF THE DISCLOSURE

The present disclosure relates in general to a fluorescence imaging apparatus, methods and systems, and more particularly, to reducing or eliminating thermal noise and ambient light noise in optical coherence tomography (OCT) and fluorescence spectroscopy.

### BACKGROUND OF THE DISCLOSURE

Optical coherence tomography (OCT) provides high-resolution, cross-sectional imaging of tissue microstructure in situ and in real-time, while fluorescence imaging, like near-infrared autofluorescence (“NIRAF”), enables visualization of molecular processes. The integration of OCT and fluorescence imaging in a single catheter provides the capability to simultaneously obtain co-localized anatomical and molecular information from a tissue such as the artery wall. For example, in “Ex. Vivo catheter-based imaging of coronary atherosclerosis using multimodality OCT and NIRAF excited at 633 nm” (Biomed Opt Express 2015, 6(4):1363-1375), an OCT-fluorescence imaging system using He: Ne excitation light for fluorescence and swept laser for OCT simultaneously through the optical fiber probe.

An MMOCT product is designed to image arteries by rotating a catheter at a high speed while performing a short linear movement distally (or in reverse). The catheter is controlled by the PIU (Patient Interface Unit) assembly which both rotates and moves the catheter with a pair of motors. The catheter is contained within a sheath while inserted into the artery. There is an event called drill through where a catheter's internal rotary components, which are attached to the motor, drill through the non-rotating external catheter sheath, which may possibly damage or pierce a patient's artery causing injury or death.

The MMOCT product contains mitigating factors which help to reduce the chance of injury. For example, the sheath wall physically prevents drill through from happening until the catheter moves forward a certain distance, in addition the proper software should monitor the state of drill through during operation. But sometimes the software may glitch or miscalculate, causing the catheter to move forward uncontrolled into the sheath wall.

Accordingly, it is particularly beneficial to devise apparatus, methods and systems for reducing or eliminating unintentional movement in the catheter in optical coherence tomography (OCT) and fluorescence spectroscopy.

### SUMMARY

Thus, to address such exemplary needs in the industry, the present disclosure teaches apparatus, systems and methods having an optical system with an optical probe for measuring a sample; and at least one motor for manipulating the optical probe, wherein the optical system comprises a circuit in communication with the motor, such that the circuit is configured to restrict movement of the motor upon a trigger.

In one embodiment, the optical system has circuitry that is configured to detect motor speed and/or movement. In yet another embodiment, the circuit to detect motor speed is based on an encoder signal from the motor.

Further embodiment include the circuit to detect motor speed is a frequency to voltage converter circuit based on a motor's encoder's index signal.

In yet another variation, the circuit to detect motor movement is based on a two encoder channel inputs.

Further embodiments, teach the circuit to detect motor movement is based on counting directional pulses of the motor.

The optical system may further comprise an optical coherence tomography.

In additional iterations of the optical system, the circuit to detect motor speed and/or movement is triggered upon a predetermined threshold.

Further embodiment may teach the motor being restricted only when traveling in a forward direction with respect to the optical system.

The subject disclosure also teaches methods for operating an optical system, including an optical system comprising: an optical probe for measuring a sample; and at least one motor for manipulating the optical probe, wherein the optical system comprises a circuit in communication with the motor, wherein the method includes: operating the at least one motor in the optical system, monitoring the circuit in the optical system for a predetermined trigger, and restricting movement of the motor once the predetermined trigger is reached.

In one embodiment the circuit is configured to detect motor speed and/or movement.

In yet another embodiment, the circuit to detect motor speed is based on an encoder signal from the motor. Furthermore, the circuit to detect motor speed is a frequency to voltage converter circuit based on a motor's encoder's index signal.

In yet another embodiment, the circuit to detect motor movement is based on a two encoder channel inputs.

Furthermore, the subject innovation teaches that the circuit to detect motor movement is based on counting directional pulses of the motor.

In yet another embodiment, the optical system further comprises optical coherence tomography.

In further embodiment, the circuit to detect motor speed and/or movement is triggered upon a predetermined threshold.

In additional embodiments, the motor is restricted only when traveling in a forward direction with respect to the optical system.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present disclosure will become apparent from the following detailed description when taken in conjunction with the accompanying figures showing illustrative embodiments of the present invention.

FIG. 1 is a schematic diagram of OCT and fluorescence multi-modality system, according to one or more embodiment of the subject apparatus, method or system.

FIG. 2 provides a cut-away side perspective view of an exemplary catheter, according to one or more embodiments of the subject apparatus, method or system.

FIG. 3 depicts an exemplary block diagram of the subject innovation, according to one or more embodiment of the subject apparatus, method or system.

FIG. 4 depicts a full circuit/electronic schematic of the subject innovation, according to one or more embodiment of the subject apparatus, method or system.

FIG. 5 provides a high speed detection circuit/electronic schematic of the subject innovation, according to one or more embodiment of the subject apparatus, method or system.

FIG. 6 is a forward detection and pulse generator circuit/electronic schematic of the subject innovation, according to one or more embodiment of the subject apparatus, method or system.

FIG. 7 provides an exemplary forward pulse counter and comparator circuit/electronic schematic of the subject innovation, according to one or more embodiment of the subject apparatus, method or system.

FIG. 8 is a combinatorial logic circuit/electronic schematic of the subject innovation, according to one or more embodiment of the subject apparatus, method or system.

FIG. 9 is a graph depicting the timing diagram of a high speed detection circuit of the subject innovation, according to one or more embodiment of the subject apparatus, method or system.

FIG. 10 provides a graph of a timing diagram of the entire forward detection circuit/electronic of the subject innovation, according to one or more embodiment of the subject apparatus, method or system.

FIG. 11 is a graph of a trigger output alignment with forward detection circuit/electronic of the subject innovation, according to one or more embodiment of the subject apparatus, method or system.

FIG. 12 is a schematic for a frequency to voltage converter, according to one or more embodiment of the subject apparatus, method or system.

FIG. 13 provides block diagrams of the subject innovation, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

Fiber optic catheters and endoscopes have been developed to gain access to internal organs for the purpose of medical prognosis, evaluation, and treatment. For example in the cardiology, OCT (optical coherence tomography), white light back-reflection, NIRS (near infrared spectroscopy) and fluorescence technology have been developed to see structural and/or molecular images of vessels with the use of a catheter. The catheter, which comprises a sheath and an optical probe, is navigated into a coronary artery, near the point of interest. In order to acquire cross-sectional images of tubes and cavities such as vessels, esophagus and nasal cavity, the optical probe is rotated with a fiber optic rotary joint (FORJ). In addition, the optical probe may be simultaneously translated longitudinally during the rotation so that helical scanning pattern images are obtained, providing a three-dimensional rendering of the cavity. This translation is most commonly performed by pulling the tip of the probe back towards the proximal end of the cavity, hence earning the common name ‘pullback’.

Imaging of coronary arteries by intravascular OCT and fluorescence system is described in a first embodiment of the subject innovation. In particular, the system is able to obtain reliable florescence signals using the subject noise reduction method(s).

FIG. 1 shows an exemplary OCT and fluorescence multi-modality system 10. Here, an OCT light with a wavelength of around 1.3 μm, from an OCT light source 12, is delivered and split into a reference arm 16 and a sample arm 18 with a splitter 14. A reference beam 20 is reflected from a reference mirror 22 in the reference arm 16 while a sample beam 24 is reflected and/or scattered from a sample (not shown) through a PIU 26 (patient interface unit) and a catheter 28 in the sample arm 24. Fibers of the PIU 26 and catheter 28 are made of a DCF (double clad fiber). The OCT light 12 illuminates the sample through the core of DCF, and scattered light from the sample are collected and delivered back to the circulator 30 of an OCT interferometer via the PIU 26. The collected light is combined with the reference beam 20 at the combiner 32 and generates interference patterns. The output of the interferometer is detected with the OCT detectors 34 such as photodiodes or multi-array cameras. Then the signals are transferred to a computer 36 to perform signal processing to generate OCT images. The interference patterns are generated only when the path length of the sample arm 18 matches that of the reference arm 16 to within the coherence length of the light source. An excitation light with wavelength of 0.635 um, from a fluorescence light source 38, is delivered to the sample through the PIU 26 and the catheter 28. The PIU 26 comprises a free space beam combiner so that the excitation light couples into the common DCF with OCT. The excitation light illuminates the sample from the distal end of the optical probe in the catheter 28. The sample emits auto-fluorescence with broadband wavelengths of 0.65-0.90 um, and auto-fluorescence are collected with the catheter 28 and delivered to a fluorescence detector 40 via the PIU 26.

The PIU 26 comprises a free space beam combiner, a FORJ (Fiber Optic Rotary Joint), a rotational motor and a translation motorized stage, and a catheter connector. The FORJ allows uninterrupted transmission of an optical signal while rotating the double clad fiber on the left side along the fiber axis. The FORJ has a free space optical beam coupler to separate a rotor and a stator. The rotator comprises a double clad fiber with a lens to make a collimated beam. The rotor is connected to the optical probe, and the stator is connected to the optical sub-systems. The rotational motor delivers the torque to the rotor. In addition, the translation motorized stage may be used for pullback. A catheter connector is connected to the catheter.

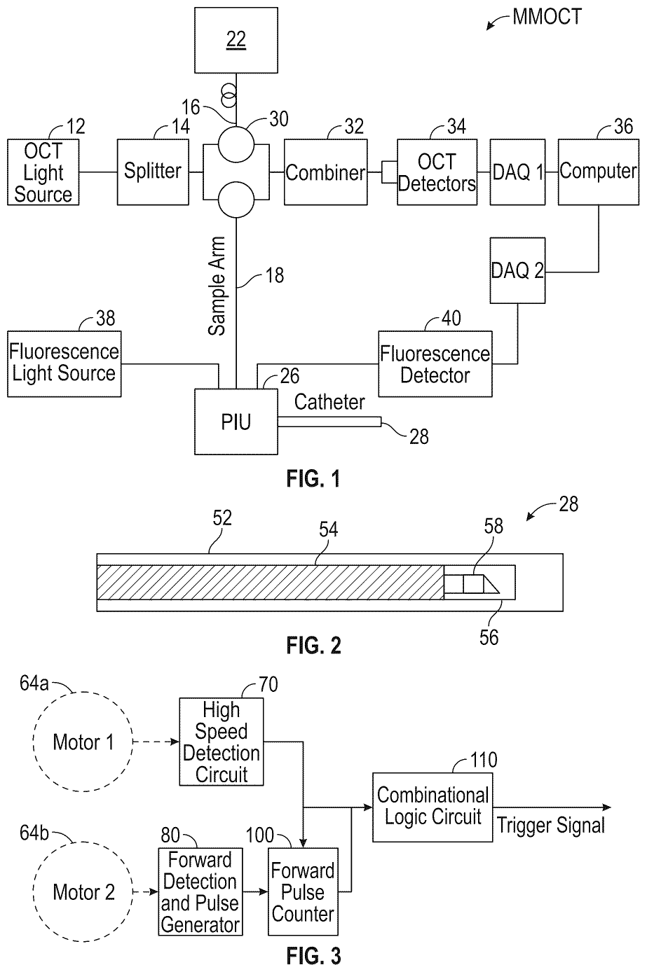

The catheter 28, which comprises a sheath 52, a coil 54, a protector 56 and an optical probe 58, is connected to the PIU 26, as shown in FIG. 2. The optical probe 58 comprises an optical fiber connector, an optical fiber and a distal lens. The optical fiber connector is used to engage with the PIU, and to deliver light to the distal lens. The distal lens is utilized in shaping the optical beam and to illuminate light to the sample, and to collect light from the sample efficiently.

The coil 54 delivers the torque from the proximal end to the distal end by a rotational motor in the PIU 26. There is a mirror 60 at the distal end so that the light beam is deflected outward, at an angle of about 90 degrees to the length of the catheter 28. The coil 54 is fixed with the optical probe 58 so that a distal tip of the optical probe 58 also spins to see omnidirectional views of the inner surface of hollow organs such as vessels. The optical probe 58 comprises a fiber connector at the proximal end, a double clad fiber, and a lens at the distal end. The fiber connector is connected with the PIU 26. The double clad fiber is used to transmit and collect OCT light through the core, and to collect Raman and/or fluorescence from sample through the clad. The lens focuses and collects light to and/or from the sample. The scattered light through the clad is relatively higher than that through the core because the size of the core is much smaller than the clad.

As mentioned earlier, the focus of this disclosure is an apparatus, methods and systems to mitigate or eliminate injuries when incorporating a catheter 28 drill through, which is an event that requires specific movements from the catheter 28 to occur. These movements in the catheter 28 give rise to incidents in which a trigger signal 50 should be generated, as seen in Table 1. As such, the catheter 28 is allowed to rotate at any speed only while moving in reverse (or distally), but when moving forward (or proximally) must rotate slowly to prevent drill through from occurring.

TABLE 1
Drill Through Events

| Movement Case | Drill through event? |
|---|---|
| Motors stopped | No |
| Low speed reverse/distal | No |
| High speed reverse/distal | No |
| Low speed forward/proximal | No |
| High speed forward/proximal | Yes |

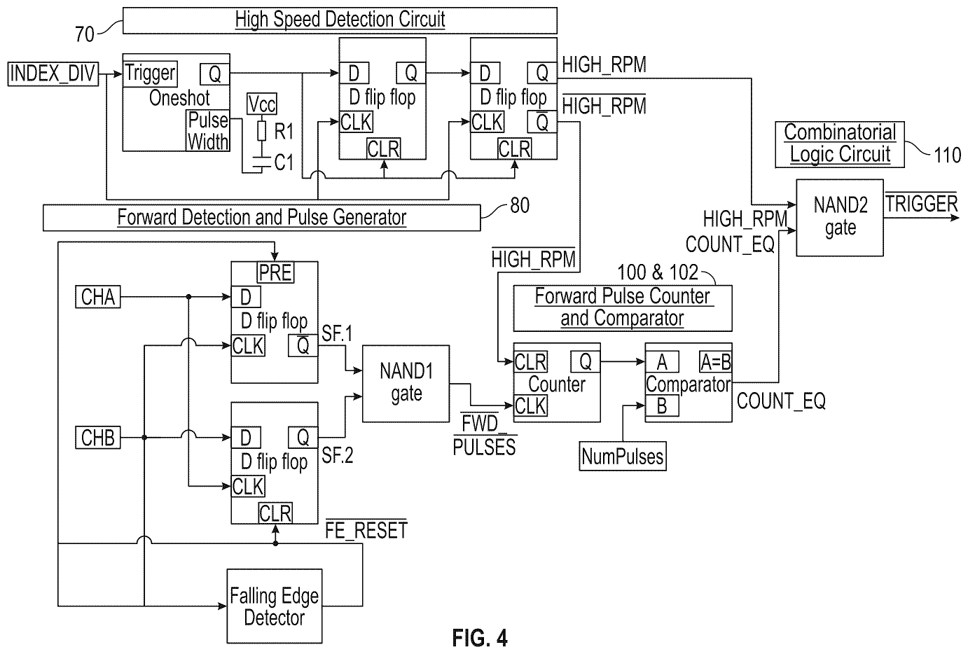

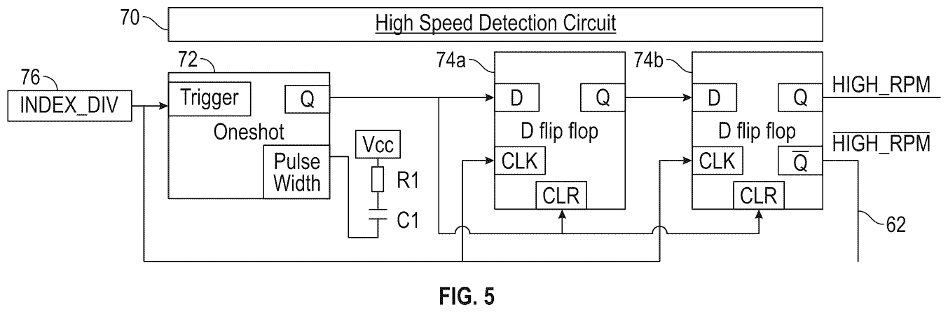

To create a trigger signal 50 that can be used by subsequent circuits, it is proposed to electrically detect all cases of movement by the catheter 28, but only generate a trigger signal 50 for forward movement and/or high speed rotation over a threshold for the catheter 28. The trigger signal 50 could be used in different ways, e.g. cut power to the motor(s) 64 or notify the processor 66, which is a software solution. Accessing the electronics provides a fast and direct way to monitor the motors 64 for the catheter 28 for a safety critical event. A hardware solution may also, or substitutionally, be utilized, which may be faster and considered more reliable than a software solution. A high level design of the entire circuit 68 can been seen in FIGS. 3 and 4, with detailed schematics of each element of the circuit presented in FIGS. 5-8.

### High Speed Detection Circuit

FIG. 5 provides details of the high-speed detection circuit, which makes up a portion of the overall circuit 68. The high-speed detection circuit 70 is utilized to detect high speed over a threshold by incorporating a retriggerable multivibrator, or oneshot 72, and pair of D-type flip-flops, 74a and 74b, respectively, are used which input any of the rotary motor's 64 encoder signals. For faster response time, one of the encoder channels is used since it is a division of the full revolution indicator, or INDEX 76. The timer on the oneshot 72 is set to the desired threshold through C1 and R1. As the rotary motor 64 spins at low speeds, the oneshot 72 outputs a short pulse that coincides with the encoder 78. At high speeds, the interval between pulses shorten until the pulses overlap and appear as one continuous pulse. This output pulse is fed to the pair of flip flops 74a and 74b. The first flip flop 74a inputs the oneshot 72 output to its DATA and CLEAR inputs and is clocked by the encoder signal. When the oneshot 72 outputs short pulses, it enables the flip-flops 74a, 74b for a short period but clears their outputs so no data is clocked. When the oneshot 72 is over threshold and outputting a continuous pulse, the D flip-flops 74a, 74b operate as normal and latch the continuous pulse on every encoder pulse. They will output a constant high until the motor speed falls below threshold, which results in the flip flops 74a and 74b being cleared as the high speed state is no longer valid. The second flip flop 74b is only to prevent metastability. The output of the flip-flops 74a and 74b, delay the actual motor's encoder 78 by two motor revolutions, resulting in a response time of two encoder 78 clock cycles.

### Forward Detection and Pulse Generator

The forward detection element 80 is comprised of 2 circuits 82 and 84. The forward detector 82 generate pulses when the linear motor is moving forward and the pulse generator 84 is used to count the amount of pulses before confirming forward motion.

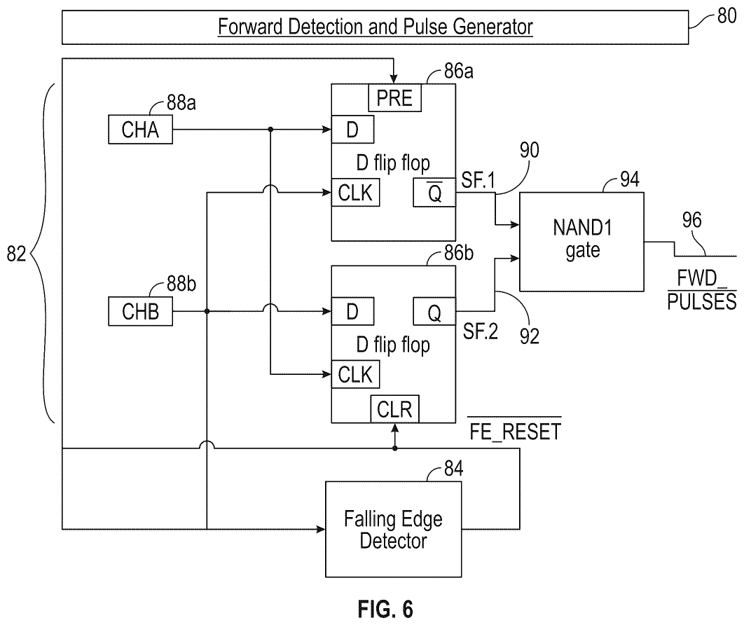

As seen in FIG. 6, the forward detector 82 is made of two D-type flip-flops, 86a and 86b, that detect different states of a forward movement. When a pair of encoders 88a and 88b (channel A and B) are used to track direction of a motor 64, there are 4 states per direction that are possible as seen in Table 2 below. The flip-flops 86a and 86b in the circuit each detect forward states SF.1 90 and SF.2 92 and feed to the NAND1 94 gate. When both states SF.1 90 and SF.2 92 are detected the output of the NAND gate goes low at the ˜FWD_PULSES output 96. The subsequent counter circuit only counts pulse edges, but the flip-flops 86a, 86b, are latches and thus maintain their output once triggered. To prevent this, the flip-flops 86a and 86b, need to be reset for each encoder pulse. Due to the state logic, one flip-flop is PRESET while the other is CLEARED. These inputs are fed by a falling edge pulse generator 84. This pulse generator 84 is fed by channel B of the encoder 88b. Whenever encoder channel B 88b generates a normal pulse from motor 64 movement, the falling edge of that pulse will produce a short negative pulse that activates the reset inputs of both flip-flops 86a and 86b. When fed by the train of encoder pulses, the circuit generates a series of pulses for the counter to count when the motor 64 moves forward. Two forward states are detected, the flip-flops 86a, 86b are set and in agreement, then they are reset.

TABLE 2
Encoder States

| State | Forward | | State | Reverse | |
|---|---|---|---|---|---|
| Name | Channel A | Channel B | Name | Channel A | Channel B |
| SF.1 | Low | Rising | SR.1 | Low | Falling |
| SF.2 | Rising | High | SR.2 | Rising | Low |
| SF.3 | High | Falling | SR.3 | High | Rising |
| SF.4 | Falling | Low | SR.4 | Falling | High |

### Forward Pulse Counter and Comparator

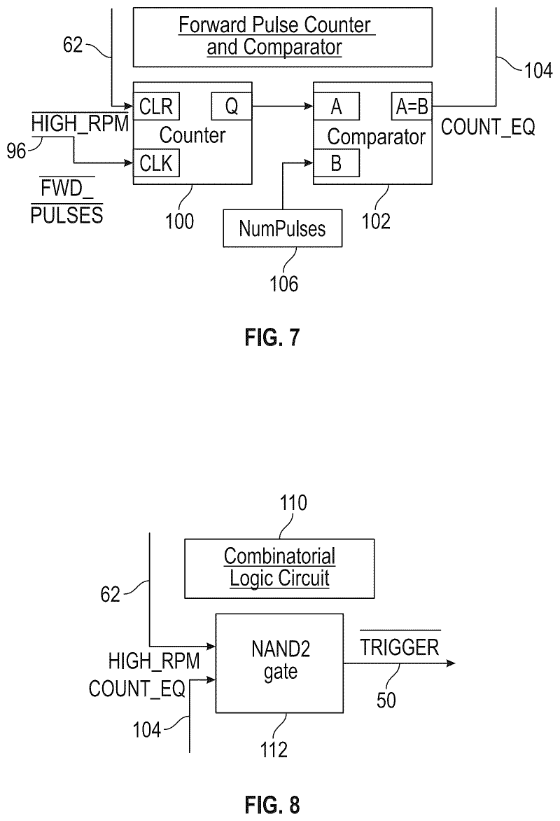

The forward pulse counter 100 and comparator 102, detailed in FIG. 7, counts a predetermined amount of pulses from the forward detection element 80 then outputs a HIGH at COUNT_EQ 104. The amount of pulses can be equated to a distance travelled with a fixed encoder. The counter 100 uses the pulse train from the forward detector 82 as its CLOCK and the HIGH_RPM output from the high speed detection circuit 70 as its CLEAR. By gating the circuit with the high speed detection circuit 70, it will only have the chance to trigger while the motor 64 is moving above the high speed threshold with no chance to trigger falsely at low speeds. Once the hardwired amount of pulses are counted, COUNT_EQ goes high.

The timing of the encoders 78, 88a and 88b, does not matter as the circuit relies on discrete states. Any speed at which the motor 64 moves will equate to the same distance as the encoder pulses are physically placed a set distance from each other on the motor 64 itself.

### Combinatorial Logic Circuit

The combinatorial logic circuit 110 is seen in FIG. 8, and is simply a NAND gate (NAND2) 112 that detects the states that indicate drill through. The configuration could be changed to an AND gate (not shown) depending on the logic required by any subsequent circuits, which would output HIGH when triggered rather than LOW. Once the high speed detection 70 and forward detection 80 circuits are triggered, the output of the logic gate triggers for as long as the drill through event is present. It will stay in this state until either of the conditions causing drill through are removed. Specifically, for MMOCT, the trigger line is used to disable power to the motors 64 so no more rotary or linear movement is allowed, and the device must be power cycled to resume.

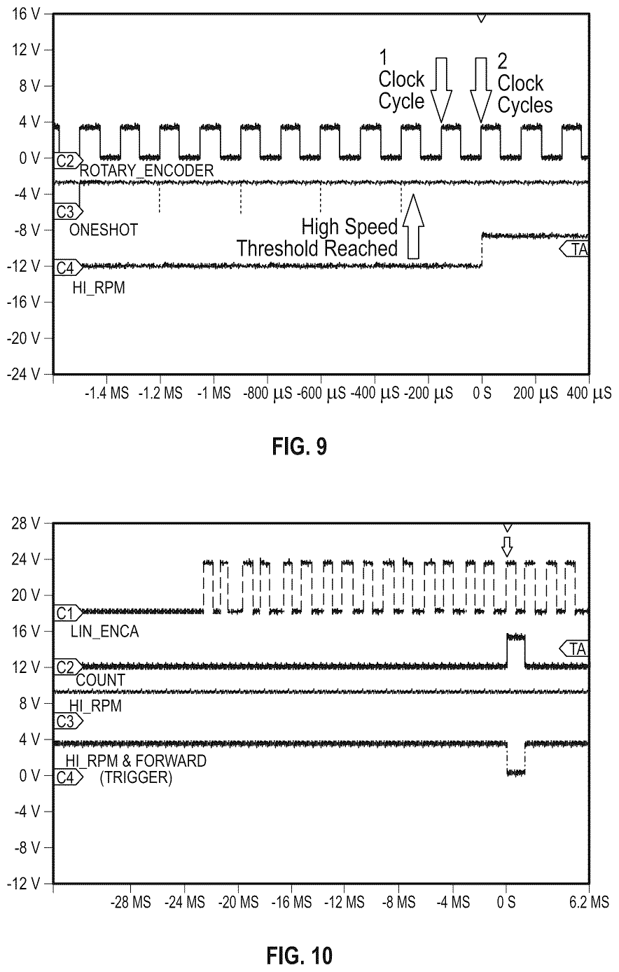

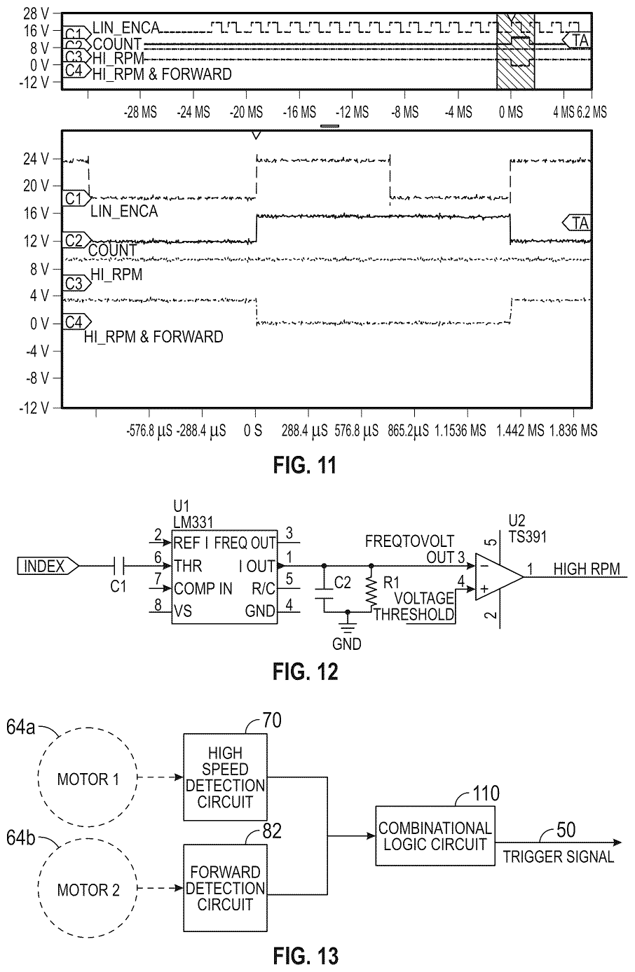

FIG. 9 shows the timing diagram for the High Speed Detection circuit 70. With FIGS. 10 and 11 showing the timing diagrams for the Forward Detector 82 and Pulse Generator 84, and Pulse Counter 100 and Comparator 102 circuits, respectively. Since the high speed and forward states cannot trip at the exact same time, the response time of the entire circuit is determined by whichever event happens last. Specifically, to prevent drill through, the device requires a maximum response time of 0.5 seconds, but is preferably enabled in 0.1 seconds or less.

The High Speed Detection circuit 70 uses one of the motor's 64 encoder channels 78 rather than the index to respond as fast as possible. The circuit will always respond within 2 clock cycles. FIG. 9 shows that as soon as the motor accelerates over the threshold, the oneshot 72 turns into a continuous pulse rather than dropping low periodically. Two clock cycles later, the HIGH_RPM signal 62 triggers, indicating the motor 64 is in the high speed state. While the circuit is adjustable, this specific timing diagram shows a threshold of 1512 RPM, or a period of 159 us. The maximum response time is approximately 318 us, or 2*159 us.

The forward detection circuit 80 requires two edges from the encoder 88a and 88b channel to create a pulse for the counter. The maximum response time of the circuit is N+1 encoder pulses, where N is the count of the pulse counter. The response is N+1 rather than N since the two encoder 88a and 88b states required to trigger a forward pulse do not have to occur sequentially. If the encoder 88 were to start while the encoder 88 was between the two states, the circuit would only detect 1 state then reset, then require another 2 states to generate a pulse.

FIG. 10 shows the circuit triggering on a count of 16 while in the high speed state on HI_RPM. After 16 pulses, COUNT_EQ 104 signal is triggered as well as the Combinatorial Logic Circuit 110. FIG. 11 shows how the output TRIGGER 50 of the combinatorial safety circuit occurs almost instantly once forward motion is detected. While this circuit was preconfigured for 16 counts, the count is adjustable to tune in time and distance travelled of the motor at the NumPulses 106 input of the comparator in FIG. 7.

### Design Iterations

FIG. 12 is provided to showcase an analog solution for high rotary speed detection that used a frequency to voltage converter to convert the rotary INDEX 76 signal 76 to analog voltage that varied based on the speed of the motor; higher speeds equate to higher voltages. While the design specifies a certain IC, any frequency to voltage converter with similar characteristics would work. The output of the frequency to voltage converter was fed to a comparator that converted the analog signal back to a digital one. Once the motor speed was fast enough and the output rose above the Voltage Threshold signal of the comparator, it would output high on the HIGH_RPM signal 62 indicating the motor 64 was in the high speed state. The response time of this design was very slow. The circuit works by using the input pulses from the INDEX signal 76 to slowly charge the RC circuit at R1 and C2, and the speed was heavily dependent on the capacitor value and motor speed. At high speeds, the INDEX signal 76 would quickly charge the output, resulting in a fast response time. At low speeds, the INDEX signal 76 train has a large period between pulses and the frequency to voltage converter takes multiple pulses to trigger. With a 33.3 ms period and 10 uF∥100 k Ω combination on C2∥R1, the response time was 2.64 seconds, almost 10 pulses, which is unacceptable for a safety critical circuit.

FIG. 13 provides a second iteration similar to the final design (seen in FIG. 4-8), using dual flip flops 74a and 74b for the high speed detection circuit 70 but only 1 flip flop 86a for the forward detection circuit 80. The benefit of the dual flip flops74a and 74b for high speed detection 70 is response time. It has a maximum response time of 2 clock cycles, which is much faster than the first iteration seen in FIG. 12. The issue is it only detects one forward state transition and does not account for motor 64 jitter, which would lead to false triggers. Jitter is when the motor 64 is settling on its final position but moves back and forth on a single encoder edge as the feedback loop within the controller settles. The motor 64 could jitter on a single edge for a number of encoder pulses after any movement. Since the circuit only identified a single pulse as forward movement, the circuit would falsely trigger after a valid pullback and shut down the system.

The second iteration does not contain a Forward Pulse Generator 84 or a Forward Pulse Counter 100 to count a minimum distance, which can be seen in block diagram. In the PIU 26, the linear motor 64 runs at full speed. There were instances where it would overshoot its final position after a pullback and have to move forward an imperceptible amount to stop at the correct position. This was not jitter but a valid, small forward movement. This version still counted 2 forward states but the motor 64 moved forward by >=2 states while still spinning at high speed, thus triggering drill through and shutting down the system.

1. An optical system comprising:
an optical probe for measuring a sample; and
at least one motor for manipulating the optical probe,
wherein the optical system comprises a circuit in communication with the motor, such that the circuit is configured to restrict movement of the motor upon a trigger.

2. The optical system of claim 1, wherein the circuit is configured to detect motor speed and/or movement.

3. The optical system of claim 2, wherein the circuit to detect motor speed is based on an encoder signal from the motor.

4. The optical system of claim 2, wherein the circuit to detect motor speed is a frequency to voltage converter circuit based on a motor's encoder's index signal.

5. The optical system of claim 2, wherein the circuit to detect motor movement is based on a two encoder channel inputs.

6. The optical system of claim 2, wherein the circuit to detect motor movement is based on counting directional pulses of the motor.

7. The optical system of claim 1, further comprising optical coherence tomography.

8. The optical system of claim 1, wherein the circuit to detect motor speed and/or movement is triggered upon a predetermined threshold.

9. The optical system of claim 1, wherein the motor is restricted only when traveling in a forward direction with respect to the optical system.

10. A method for operating an optical system comprising:
an optical system comprising:
an optical probe for measuring a sample; and
at least one motor for manipulating the optical probe, wherein the optical system comprises a circuit in communication with the motor,
the method comprising:
operating the at least one motor in the optical system,
monitoring the circuit in the optical system for a predetermined trigger,
restricting movement of the motor once the predetermined trigger is reached.

11. The method of claim 10, wherein the circuit is configured to detect motor speed and/or movement.

12. The method of claim 11, wherein the circuit to detect motor speed is based on an encoder signal from the motor.

13. The method of claim 11, wherein the circuit to detect motor speed is a frequency to voltage converter circuit based on a motor's encoder's index signal.

14. The method of claim 11, wherein the circuit to detect motor movement is based on a two encoder channel inputs.

15. The method of claim 11, wherein the circuit to detect motor movement is based on counting directional pulses of the motor.

16. The method of claim 10, further comprising optical coherence tomography.

17. The method of claim 10, wherein the circuit to detect motor speed and/or movement is triggered upon a predetermined threshold.

18. The optical system of claim 10, wherein the motor is restricted only when traveling in a forward direction with respect to the optical system.
